(19) United States
(12) Patent Application Publication
(10) Pub. No.: US 2009/0253158 A1
(43) Pub. Date: Oct. 8, 2009

(54) Lipoxygenase Enzyme Assay

(75) Inventors: John Alan Broadwater, Southbury, CT (US); Hnin Hnin Khine, Danbury, CT (US); Kristine Woodruff, Woodbury, CT (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(73) Assignee: BOEHRINGER INGELHEIM INTERNATIONAL GMBH, Ingelheim (DE)

(21) Appl. No.: 12/373,540

(22) PCT Filed: Jul. 23, 2007
(86) PCT No.: PCT/US07/74075

§ 371 Date: Jan. 13, 2009

Related U.S. Application Data
(60) Provisional application No. 60/820,390, filed on Jul. 26, 2006.

(51) Int. Cl. C12Q 1/28

(52) U.S. Cl. 435/28

(57) ABSTRACT

A method for identifying inhibitors of a lipoxygenase enzyme, the assay comprising: contacting a lipoxygenase enzyme with a test Compound, a lipoxygenase enzyme Substrate and oxygen; adding a fluorometric reagent and a peroxidase; measuring the fluorescent signal; determining the amount of enzyme inhibition by the test Compound.

### APPLICATION DATA

This application claims benefit to U.S. provisional application Ser. No. 60/820,390 filed Jul. 26, 2006.

### BACKGROUND OF THE INVENTION

1. Technical Field

This invention relates to methods and kits for lipoxygenase enzymes which catalyze the oxygen-dependent oxidation of fatty acid substrates (linoleic acid and arachidonic acid are common examples) to form hydroperoxy-fatty acid products. The methods and kits are useful for detecting inhibitors of such enzymes.

2. Background Information

Lipoxygenase enzymes catalyze the oxygen-dependent oxidation of fatty acid substrates (linoleic acid and arachidonic acid are common examples) to form hydroperoxy-fatty acid products. Enzymes have been purified from diverse organisms that display a broad range of substrate specificity and product specificity (i.e. the site of oxidation within the fatty acid).

Several assay procedures have been published in the literature but each has particular limitations that make high-throughput screening difficult. The simplest assay is the spectrophotometric monitoring of the hydroperoxy-fatty acid product; the hydroperoxy-moiety absorbs light at 234 nm and can therefore be easily monitored with a spectrophotometer. As many potential inhibitors absorb light at this wavelength, this assay format is prone to interference from the very compounds we seek. Another method of assaying for lipoxygenase activity is to monitor the consumption of oxygen using a Clark electrode; this method is neither sensitive nor amenable to high-throughput. Another assay that has been used is to determine the concentration of hydroperoxy (or the chemically-reduced hydroxy-derivatives) fatty acids by separation from the substrate on a high-performance liquid chromatography (HPLC) system (for example, Yamamoto et al. (1990) Methods in Enzymology, 186, 371-380). These assays, while accurate, are heterogeneous and time consuming and are therefore not amenable to screening large numbers of compounds. Two different calorimetric assay formats have been developed that utilize the oxidation state of the hydroperoxy product to couple product formation to color formation. Both assays are conducted in two steps and differ on the calorimetric reagent. After product has been formed, the color-forming reagent is added and the color is measured on a spectrophotometer. One assay used a xylenol orange:iron(II) complex (Waslidge et al. (1995) Anal. Biochemistry, 231, 354-358) and the second assay used hemoglobin as the catalyst) and N-benzoyl leucomethylene (Auerbach et al. (1992) Anal. Biochemistry, 201, 375-380) as the calorimetric reagent. These assays offer improved sensitivity over the direct spectrophotometric assay (˜10-fold) and improved throughput when compared to the HPLC method. However, calorimetric assays suffer from a small signal-to-background window in which to measure a signal. Kratky et al. have published a very sensitive assay of lipoxygenases based upon chemiluminescent detection (Kratky et al. (1999) Biochimica et Biophyscia Acta, 1437, 13-22). The hydroperoxy-fatty acid product of lipoxygenase is reacted with isoluminol and microperoxidase to form an electronically excited form of 4-aminophthalate that emits a photon upon its decay. Because chemiluminescence is very short lived, each individual assay must be initiated and completed before proceeding to the next assay. This process makes the assay unsuitable for a high-throughput approach.

Molecular Probes (now part of Invitrogen) has published an assay for hydrogen peroxide detection that employs Amplex Red® (N-acetyl-3,7-dihydroxyphenoxazine) or Amplex UltraRed® and uses horseradish peroxidase as the redox catalyst instead of microperoxidase (Zhou et al. (1997) Anal. Biochemistry, 253, 162-168). While they sell many kits based upon the ability to couple hydrogen peroxide with Ample Red® oxidation, they do not mention the ability use the Amplex Red® reagent to detect hydroperoxy-fatty acids nor do any of their present reagents use microperoxidase as the redox catalyst.

The present inventors have designed an assay format to enable the identification of inhibitors of lipoxygenase enzymes. This assay represents a significant advantage over previous assay formats as the sensitivity and uniqueness of the signal render the format more amenable to high-throughput screening.

### BRIEF SUMMARY OF THE INVENTION

It is therefore an object of the invention to provide a method to identify inhibitor of lipoxygenase enzymes.

It is a further object of the invention to provide a kit comprising an assay to identify inhibitor of lipoxygenase enzymes.

### BRIEF DESCRIPTION OF THE FIGURES

FIG. 1: Schematic depiction of microperoxidase catalyzing a redox reaction between the hydroperoxy-fatty acid product and the Amplex UltraRed® to generate the highly fluorescent product resorufin. The amount of resorufin is then determined using fluorescence spectroscopy.

FIG. 2: Schematic depiction of the fluorometric lipoxygenase assay that would be used to characterize the activity of a 15-lipoxygenase.

FIGS. 3-5: Inhibition of x-lipoxygenase by representative compounds of varying potencies. The y-axis is Percent of Control and the x-axis units are in microM.

### DETAILED DESCRIPTION OF THE INVENTION

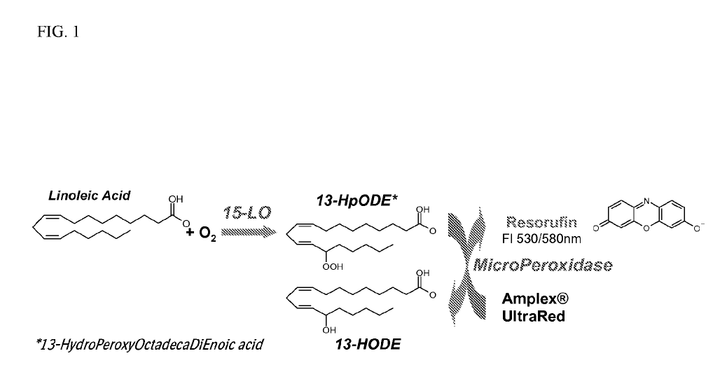

To increase assay sensitivity and retain high-throughput features (homogenous assay that can be easily automated), the present invention provides a new assay for lipoxygenase which is an improvement from the historical assays described above. After the lipoxygenase has been incubated with the fatty acid substrate (linoleic acid or arachidonic acid) and oxygen, microperoxidase (a catalyst) and Amplex UltraRed® are added. The microperoxidase catalyzes a redox reaction between the hydroperoxy-fatty acid product and the Amplex UltraRed® to generate the highly fluorescent product resorufin. The amount of resorufin is then determined using fluorescence spectroscopy (excitation at 530 nm and emission at 580 nm). See FIG. 1. This assay improves the sensitivity ˜10-fold over that observed in the colorimetric assays and generates a fluorescent signal that is both stable and free from compound interference as very few compounds fluoresce in this range.

In the broadest generic embodiment, there is provided a method for identifying inhibitors of lipoxygenase enzymes, the assay comprising:

contacting a lipoxygenase enzyme with a test compound, a lipoxygenase enzyme substrate and oxygen;
adding a fluorometric reagent and a peroxidase;
measuring the fluorescent signal;
determining the amount of enzyme inhibition by the test compound.

In second generic embodiment, there is provided a kit for determining the amount of lipoxygenase enzyme inhibition by a test compound comprising:

a lipoxygenase enzyme;
a lipoxygenase enzyme substrate;
oxygen;
a peroxidase and a fluorometric reagent.

The above kit can further contain a positive control that comprises a mock test compound. Said mock test compound having no or negligible lipoxygenase enzyme inhibition.

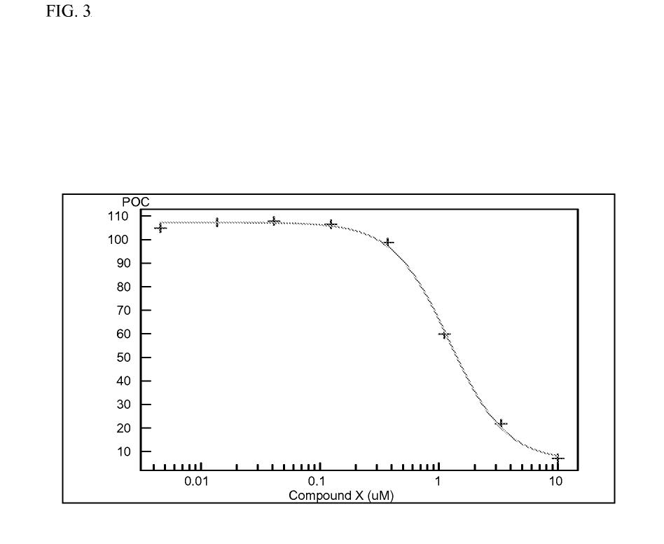

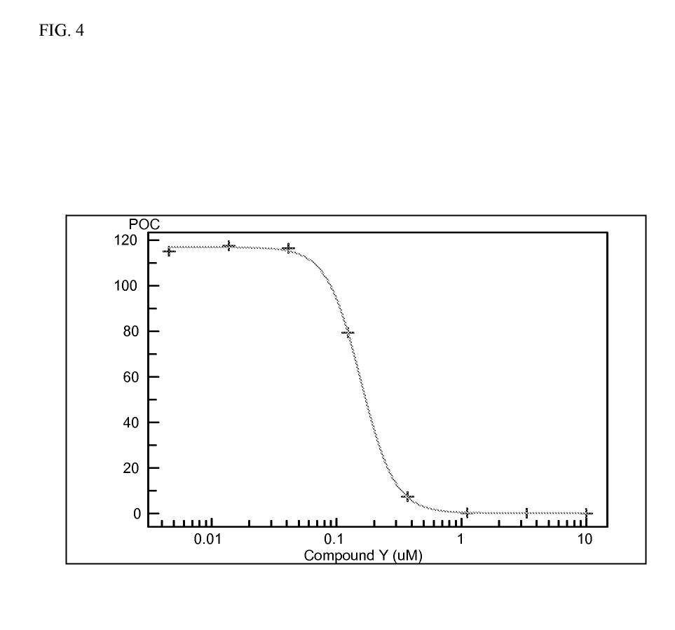

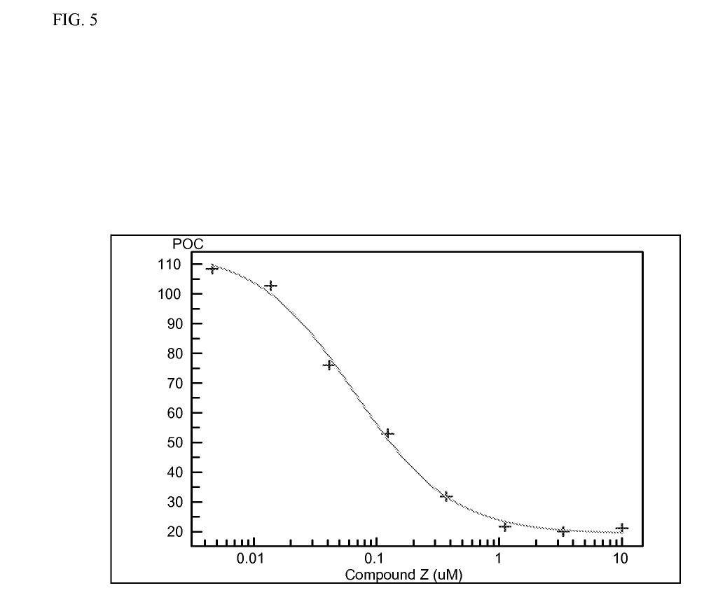

The Enzymes have been purified from diverse organisms that display a broad range of substrate specificity and product specificity. The assay as it is routinely performed is summarized in the scheme from the example section below but alterations apparent to those of ordinary skill in the art can be made. For instance, the incubation time or temperature can be adjusted but it is ideal to adjust them such that the enzyme activity is within the linear response range. The assay has been performed at various scales (cuvet, 96 or 384 well) and is expected to work at any scale required within any desired reaction vessel (e.g. polypropylene micro-plate or polystyrene cuvet). Any lipoxygenase enzyme that produces a hydroperoxy product, irrespective of stereo-specificity, is capable of being assayed by this technique, including 15-lipoxygenase from humans or soybean, 12-lipoxygenase and 5-lipoxygenase. Any substrate of the lipoxygenase enzyme can be used; this could include, but is not limited to, free fatty acids or esterified fatty acids of varying composition (e.g. arachidonic acid, linoleyl-phosphatidyl choline, low-density lipoprotein, etc.). While Amplex UltraRed® is the preferred fluorometric reagent in this protocol, Amplex Red® or any reagent that results in the production of a fluorescent molecule with similar fluorescence (excitation maximum of 530±25 nm and emission maximum of 580±25 nm) can also be used. Similarly, microperoxidase may be substituted with any peroxidase that catalyzes the reaction between the hydroperoxide product and the fluorometric reagent (i.e. Amplex UltraRed® in the preferred embodiment). The solutions used for the lipoxygenase reaction and the microperoxidase reaction may also be modified from the specified conditions so long as activity of the lipoxygenase and microperoxidase catalysts are retained. Examples of the use of this assay identify lipoxygenase inhibitors are shown in FIGS. 3-5.

### EXAMPLES

The following examples are offered to illustrate, but not to limit the present invention.

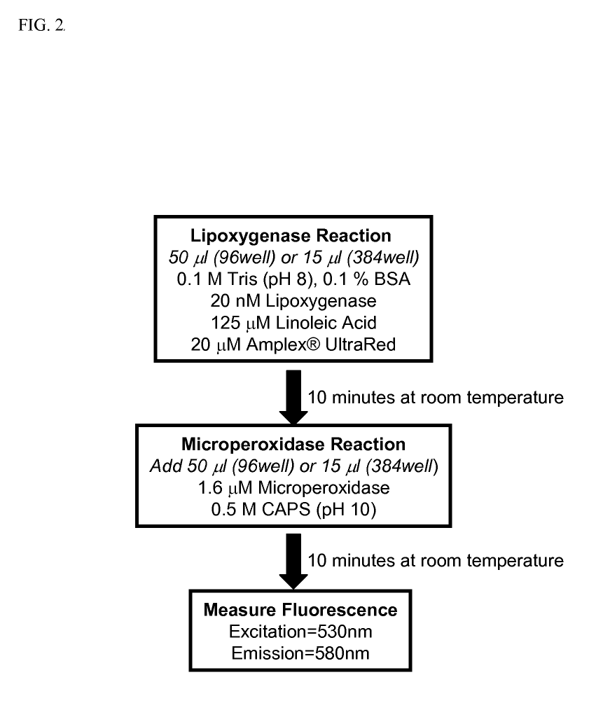

The Assay according to the invention can be performed according to the scheme shown in FIG. 2.

All referenced cited in this application are incorporated herein by reference in their entirety.

What is claimed is:

1. A method for identifying inhibitors of a lipoxygenase enzyme, the assay comprising:
contacting a lipoxygenase enzyme with a test compound, a lipoxygenase enzyme substrate and oxygen;
adding a fluorometric reagent and a peroxidase;
measuring the fluorescent signal;
determining the amount of enzyme inhibition by the test compound.

2. The method according to claim 1 wherein the lipoxygenase enzymes are chosen from 15-lipoxygenase, 12-lipoxygenase and 5-lipoxygenase.

3. The method according to claim 2 wherein
the substrate of the lipoxygenase enzyme is a free fatty acid or esterified fatty acid.

4. The method according to claim 3 wherein
the fluorometric reagent is a reagent that results in the production of a fluorescent molecule with an excitation maximum of 530±25 nm and emission maximum of 580±25 nm.

5. The method according to claim 4 wherein
the fluorometric reagent is Amplex UltraRed® and the peroxidase is microperoxidase.

6. A kit for determining the amount of lipoxygenase enzyme inhibition by a test compound comprising:
a lipoxygenase enzyme;
a lipoxygenase enzyme substrate;
oxygen;
a peroxidase and a fluorometric reagent.

7. The kit according to claim 6 further comprising a positive control.
